(19) United States
(12) Patent Application Publication
(10) Pub. No.: US 2024/0226480 A9
(43) Pub. Date: Jul. 11, 2024

(54) VALVE FOR ENDOTRACHEAL TUBE

(71) Applicant: Cody Cronk, Oklahoma City, OK (US)

(72) Inventor: Cody Cronk, Oklahoma City, OK (US)

(21) Appl. No.: 18/217,167

(22) Filed: Jun. 30, 2023

Related U.S. Application Data
(60) Provisional application No. 63/380,570, filed on Oct. 22, 2022.

(51) Int. Cl. A61M 16/04; A61M 16/20

(52) U.S. Cl. CPC A61M 16/0463; A61M 16/0402; A61M 16/045; A61M 16/20

(57) ABSTRACT

An endotracheal tube assembly comprising a tubing body and a tubing adapter. The tubing body has proximal and distal ends and a lumen extending therebetween. The tubing adapter comprises an adapter body and a valve member. The adapter body has first and second connector portions defining first and second connector flow passages, respectively. The adapter body further defines a valve chamber in fluid communication with the first and second connector flow passages. The first connector portion is matingly engaged with the proximal end of the tubing body. The valve member has a flow passage extending therethrough, is positioned in the valve chamber, and is movable between an open position, wherein the flow passage is aligned with the first and second connector flow passages, and a closed position, wherein the flow passage is misaligned with the first and second connector flow passages.

### CROSS-REFERENCE TO RELATED APPLICATIONS

This application claims the benefit of U.S. Provisional Patent Application No. 63/380,570, which was filed on Oct. 22, 2022, the contents of which are incorporated herein by reference in their entirety.

### BACKGROUND

Endotracheal tubes are commonly used in medical settings to establish and maintain an artificial airway and facilitate mechanical ventilation for patients with compromised respiratory function. An endotracheal tube is inserted into a patient's trachea, typically through the mouth or nose, and extends down into the patient's airway. Such an endotracheal tube is generally equipped with a connector or adapter inserted into a proximal end of the endotracheal tube that enables connection to a mechanical ventilator for delivering oxygen and assisting breathing.

While the current design and function of endotracheal tubes and their associated connectors or adapters have provided significant advancements in patient care, there are challenges and limitations related to their use. One particular concern arises when intubated patients require high levels of peak end respiratory pressure (PEEP), such as patients with acute respiratory distress syndrome (ARDS). Maintaining adequate PEEP levels is essential for sustaining alveolar recruitment and improving oxygenation in such patients. When a mechanical ventilator is connected or disconnected to an endotracheal tube inserted into a patient's airway, a transient loss of positive pressure occurs, which may cause alveolar derecruitment and compromise the patient's respiratory status. Furthermore, patients at risk for alveolar derecruitment due to other conditions or factors may also face similar challenges during the connection or disconnection of a mechanical ventilator.

Accordingly, there is a need for an improved solution that addresses the issues mentioned above associated with the current design of endotracheal tubes and their connectors.

### SUMMARY OF THE DISCLOSURE

The need for a solution that addresses the issues associated with the current design of endotracheal tubes and their connectors is solved by the endotracheal tube assembly disclosed herein. In one aspect, the present disclosure relates to an endotracheal tube assembly, comprising: a tubing body having a proximal end, a distal end, and a lumen extending through an entirety of the tubing body between the proximal end and the distal end; and a tubing adapter, comprising: an adapter body having a first connector portion defining a first connector flow passage having a first length and a second connector portion defining a second connector flow passage having a second length, the adapter body defining a valve chamber in fluid communication with the first connector flow passage and the second connector flow passage, the first connector flow passage being enclosed along an entirety of the first length, the first connector portion matingly engaged with the proximal end of the tubing body so the adapter body extends from the proximal end of the tubing body and the first connector flow passage is in fluid communication with the lumen of the tubing body; and a valve member having a flow passage extending therethrough, the valve member positioned in the valve chamber and movable between an open position, wherein the flow passage of the valve member is aligned with the first connector flow passage and the second connector flow passage to provide fluid communication between the first connector flow passage and the second connector flow passage, and a closed position, wherein the flow passage of the valve member is misaligned with the first connector flow passage and the second connector flow passage to fluidically seal the first connector flow passage from the second connector flow passage and thereby prevent fluid flow through the adapter body between the first connector flow passage and the second connector flow passage.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale, or in schematic in the interest of clarity and conciseness. Not every component may be labeled in every drawing. Like reference numerals in the figures may represent and refer to the same or similar element or function. In the drawings:

FIG. 1 is a perspective view of an exemplary endotracheal tube assembly constructed in accordance with the present disclosure.

FIG. 2 is a perspective view of an exemplary tubing adapter of the endotracheal tube assembly shown in FIG. 1.

FIG. 3 is a perspective view of an exemplary adapter body of the tubing adapter shown in FIG. 2.

FIG. 4 is a cross-sectional view of the adapter body taken along line 4-4 of FIG. 3.

FIG. 5 is a perspective view of an exemplary valve member of the tubing adapter shown in FIG. 2.

FIG. 6 is a front view of the valve member shown in FIG. 5.

FIG. 7 is a cross-sectional view of the valve member taken along line 7-7 of FIG. 5.

FIG. 8A is a cross-sectional view of the tubing adapter taken along line 8-8 of FIG. 2, wherein the valve member is shown in an open position.

FIG. 8B is a cross-sectional view of the tubing adapter shown in FIG. 2, wherein the valve member is shown in a closed position.

### DETAILED DESCRIPTION OF EXEMPLARY EMBODIMENTS

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein are for the purpose of description and should not be regarded as limiting.

Headings are provided for convenience only and are not to be construed to limit the invention in any manner. Embodiments illustrated under any heading or in any portion of the disclosure may be combined with embodiments illustrated under the same or any other heading or portion of the disclosure. Any combination of the elements described herein in all possible variations thereof is encompassed by the invention unless otherwise indicated herein or otherwise clearly contradicted by context.

Unless otherwise required by context, singular terms shall include pluralities, and plural terms shall include the singular, except that the term “plurality,” as used herein, does not include the singular.

All patents or published patent applications referenced in any portion of this application are herein expressly incorporated by reference in their entirety to the same extent as if each individual patent or publication was specifically and individually indicated to be incorporated by reference.

All assemblies, systems, kits, and/or methods disclosed herein can be made and executed without undue experimentation in light of the present disclosure. Where a method claim does not explicitly state in the claims or description that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of embodiments described in the specification.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” The term “plurality” refers to “two or more.”

The use of the term “at least one” will be understood to include one as well as any quantity of more than one. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The term “or” in the claims means an inclusive “and/or” unless explicitly indicated to refer to alternatives only or unless the alternatives are mutually exclusive.

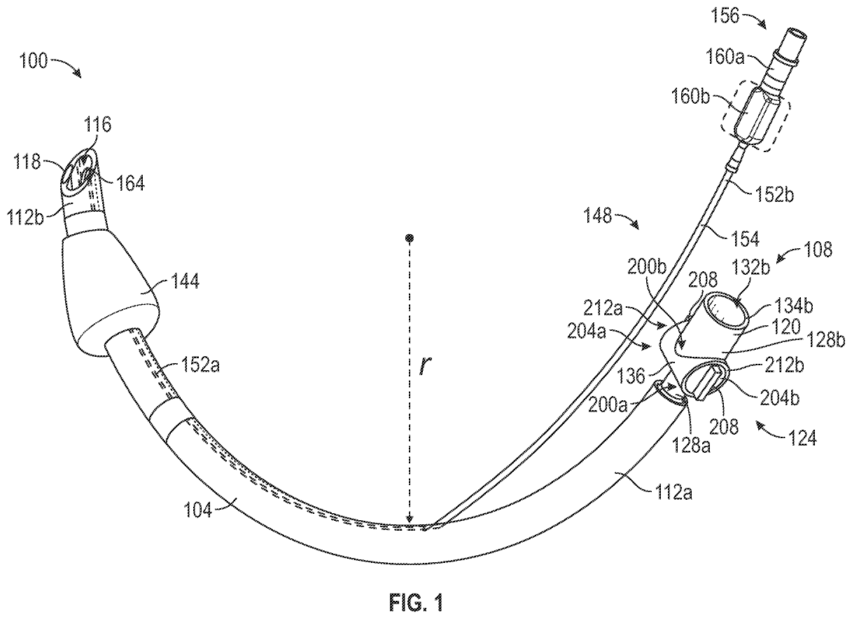

Referring now to the drawings, and in particular to FIG. 1, shown therein is a perspective view of an exemplary endotracheal tube assembly 100 (hereinafter the “assembly 100”) constructed in accordance with the present disclosure. The assembly 100 may comprise a tubing body 104 and a tubing adapter 108.

The tubing body 104 has a proximal end 112a, a distal end 112b, and a lumen 116 extending through an entirety of the tubing body 104 between the proximal end 112a and the distal end 112b, and the tubing adapter 108 is disposed at the proximal end 112a of the tubing body 104. The distal end 112b of the tubing body 104 may define a bevel 118 to facilitate insertion of the assembly 100 through the vocal cords of a patient and to improve visualization ahead of the distal end 112b of the tubing body 104.

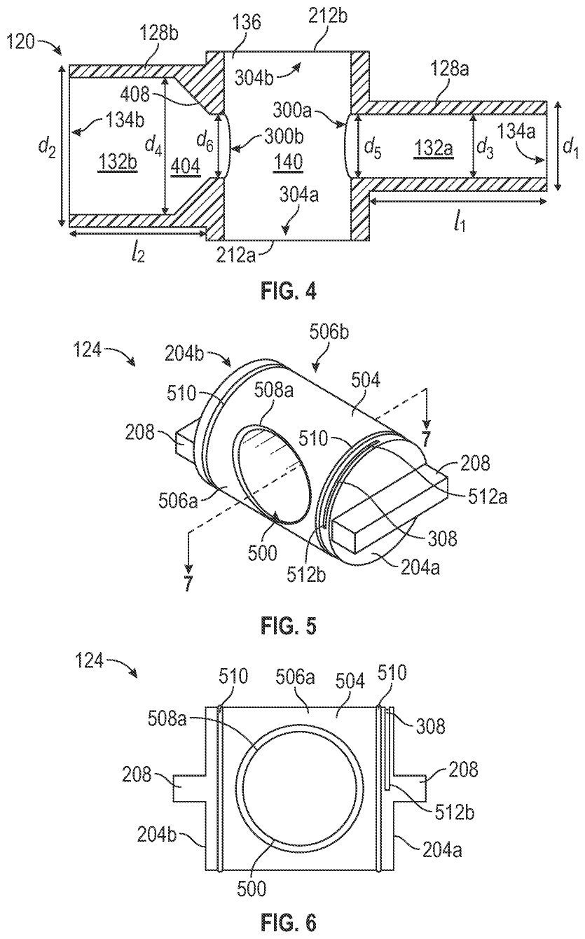

As discussed further below, the tubing adapter 108 includes an adapter body 120 and a valve member 124. The adapter body 120 has a first connector portion 128a defining a first connector flow passage 132a (shown in FIG. 4) having a first opening 134a (shown in FIG. 4), and a second connector portion 128b defining a second connector flow passage 132b having a second opening 134b. The first connector portion 128a is matingly engaged with the proximal end 112a of the tubing body 104, so the adapter body 120 extends outwardly from the proximal end 112a of the tubing body 104, and the first connector flow passage 132a is in fluid communication with the lumen 116 of the tubing body 104.

The adapter body 120 has a valve portion 136 disposed between the first connector portion 128a and the second connector portion 128b. The valve portion 136 defines a valve chamber 140 (shown in FIG. 3), wherein the valve member 124 is positioned in the valve chamber 140 and rotatable between a first position (i.e., an open position), as shown in FIG. 1, and a second position (i.e., a closed position) (shown in FIG. 8B).

As shown in FIG. 1, the assembly 100 may further include an inflatable cuff 144 positioned adjacent to the distal end 112b of the tubing body 104 and a pilot line 148 having a cuff end 152a, a lock end 152b, and a pilot lumen 154 extending between the cuff end 152a and the lock end 152b. The cuff end 152a of the pilot line 148 is coupled to the inflatable cuff 144, and the lock end 152b of the pilot line 148 is connected to a fitting 156 (i.e., a pilot balloon) disposed adjacent to the lock end 152b.

The fitting 156 may have a connector portion 160a and a balloon portion 160b. The connector portion 160a may be sized and dimensioned to be coupled to a syringe and configured only to allow airflow in one direction. In some implementations, the connector portion 160a is a female luer lock fitting. The balloon portion 160b may be biased to a first position (i.e., a deflated position), as shown in FIG. 1, and may be movable into a second position (i.e., an inflated position), as shown by the dotted lines in FIG. 1. The fitting 156 may be operable to monitor the structural integrity of the inflatable cuff 144.

The tubing body 104 may have an opening 164 (commonly referred to as a “Murphy eye”) adjacent to the distal end 112b of the tubing body 104. The opening 164 is in fluid communication with the lumen 116 of the tubing body 104. The tubing body 104 may be provided with a curvature to accommodate an upper airway of a patient. In some implementations, the curvature has a radius of 140 millimeters (mm)±20 mm; however, in other implementations, the curvature may have a radius more or less than 140 mm±20 mm.

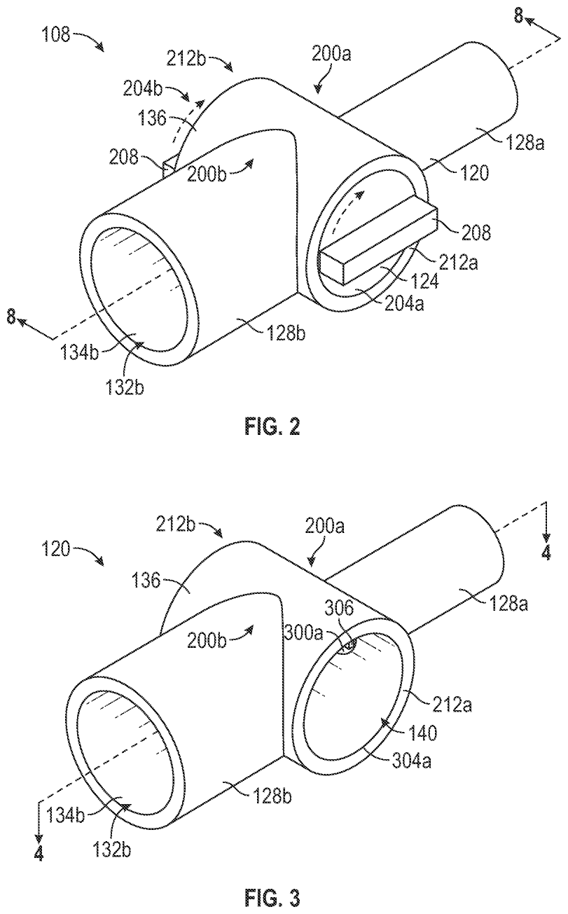

Referring now to FIG. 2, shown therein is a perspective view of the tubing adapter 108 shown in FIG. 1. As shown in FIG. 2, the valve portion 136 of the adapter body 120 has a first side 200a and a second side 200b opposite the first side 200a. The first connector portion 128a of the adapter body 120 extends outwardly from the first side 200a of the valve portion 136, and the second connector portion 128b extends outwardly from the second side 200b of the valve portion 136.

In some implementations, as shown in FIG. 2, the first connector portion 128a, the second connector portion 128b, and the valve portion 136 each have a cylindrical shape; however, in other implementations, one or more of the first connector portion 128a, the second connector portion 128b, and the valve portion 136 may have a shape other than a cylindrical shape, such as a prismatic shape, for example.

The valve member 124 has a first end 204a and a second end 204b opposite the first end 204a. Similarly, the valve portion 136 of the adapter body 120 has a first side 212a and a second side 212b opposite the first side 212a.

The valve member 124 may have one or more grip 208 (hereinafter the “grips 208”) extending outwardly from one or more of the first end 204a and the second end 204b. While the implementation of the valve member 124 shown in FIG. 2 has two grips 208, other implementations may have more or less than two grips 208. By grasping one or more of the grips 208, a user may rotate the valve member 124 in the valve chamber 140 (shown in FIG. 3) to move the valve member 124 between the first position (i.e., the open position), as shown in FIG. 2, and the second position (i.e., the closed position) (shown in FIG. 8B).

Referring now to FIG. 3, shown therein is a perspective view of the adapter body 120 of the tubing adapter 108 shown in FIG. 2. As shown in FIG. 3, the valve chamber 140 defined by the valve portion 136 may extend between the first side 212a of the valve portion 136 and the second side 212b of the valve portion 136. The valve portion 136 defines a first opening 300a in fluid communication with the first connector flow passage 132a and a second opening 300b (shown in FIG. 4) in fluid communication with the second connector flow passage 132b. Further, the valve portion 136 defines a third opening 304a on the first side 212a of the valve portion 136 and a fourth opening 304b (shown in FIG. 4) on the second side 212b of the valve portion 136.

As described further below, the adapter body 120 may have a protrusion 306 extending inwardly from the valve portion 136 into the valve chamber 140. The protrusion 306 is sized and dimensioned to engage a channel 308 (shown in FIG. 5) formed in the valve member 124.

Referring now to FIG. 4, shown therein is a cross-sectional view of the adapter body 120 shown in FIG. 3, taken along line 4-4. As referenced above, the first connector portion 128a defines the first connector flow passage 132a, and the second connector portion 128b defines the second connector flow passage 132b. Further, the valve portion 136 defines the first opening 300a in fluid communication with the first connector flow passage 132a and the second opening 300b in fluid communication with the second connector flow passage 132b.

As shown in FIG. 4, the first connector portion 128a may have an outer diameter d₁, the second connector portion 128b may have an outer diameter d₂, the first connector flow passage 132a may have an inner diameter d₃, the second connector flow passage 132b may have an inner diameter d₄, the first opening 300a may have an inner diameter d₅, and the second opening 400b may have an inner diameter d₆. The outer diameter d₁ of the first connector portion 128a may be less than the outer diameter d₂ of the second connector portion 128b. The inner diameter d₃ of the first connector flow passage 132a may be equal to the inner diameter d₅ of the first opening 300a and the inner diameter d₆ of the second opening 300b and may be less than the inner diameter d₄ of the second connector flow passage 132b.

The adapter body 120 may define a tapered chamber 404 between the second connector flow passage 132b and the second opening 300b with the tapered chamber 404 in fluid communication with the second connector flow passage 132b and the second opening 300b. The tapered chamber 404 may have a tapered wall 408 joining the second connector flow passage 132b with the second opening 300b.

In some implementations, the outer diameter d₂ of the second connector portion 128b is 15 mm; however, in other implementations, the outer diameter d₂ of the second connector portion 128b may be more or less than 15 mm.

As further shown in FIG. 4, the first connector portion 128a and the first connector flow passage 132a may have a first length l₁, and the second connector portion 128b and the second connector flow passage 132b may have a second length l₂. One of more of the first connector flow passage 132a and the second connector flow passage 132b may be enclosed along an entirety of the first length l₁ and the second length l₂, respectively.

In some implementations, the first connector flow passage 132a is axially aligned with the second connector flow passage 132b; however, in other implementations, the first connector flow passage 132a is not axially aligned with the second connector flow passage 132b.

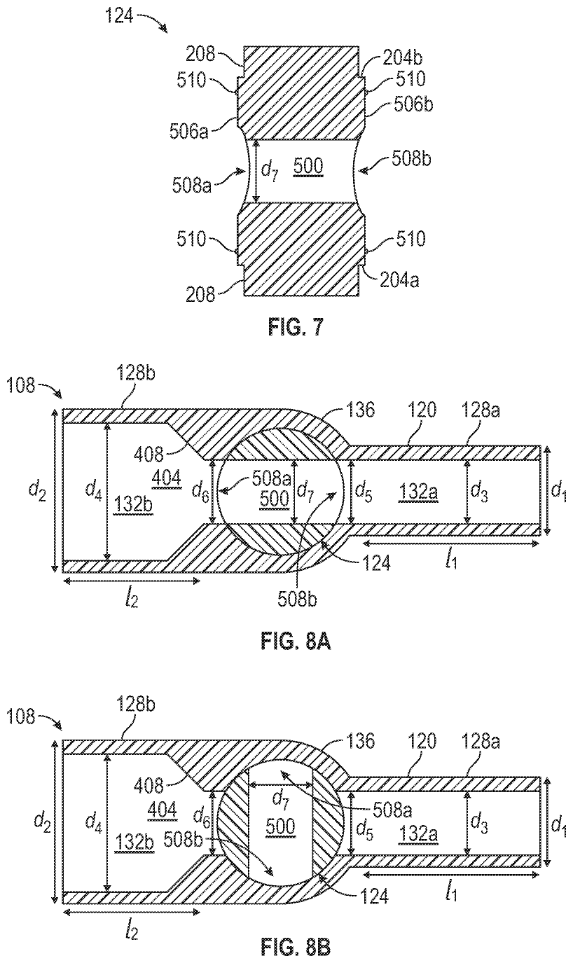

Referring now to FIGS. 5-6, shown therein are a perspective view and a front view, respectively, of the valve member 124 of the tubing adapter 108 shown in FIG. 2. As shown in FIGS. 5-6, the valve member 124 has a valve flow passage 500 extending therethrough. The valve member 124 may have a cylindrical surface 504 extending between the first end 204a and the second end 204b. The cylindrical surface 504 has a first side 506a and a second side 506b opposite the first side 506a. The valve member 124 defines a first opening 508a on the first side 506a in fluid communication with the valve flow passage 500 and a second opening 508b (shown in FIG. 7) on the second side 506b in fluid communication with the valve flow passage 500.

As referenced above, the valve member 124 may have a channel 308 formed therein sized and dimensioned to engage the protrusion 306 of the adapter body 120 shown in FIG. 3. The channel 308 may engage the protrusion 306 such that rotation of the valve member 124 is restricted to a quarter turn. Further, the valve member 124 may be provided with one or more O-ring 510 (hereinafter the “O-rings 510”) surrounding the cylindrical surface 504.

The valve member 124 may have a first locking mechanism 512a configured to secure the valve member 124 in the first position (i.e., the open position) and/or a second locking mechanism 512b configured to secure the valve member 124 in the second position (i.e., the closed position). In the implementation shown in FIGS. 5 and 6, the first locking mechanism 512a and the second locking mechanism 512b are depicted as notches in the channel 308.

Referring now to FIG. 7, shown therein is a cross-sectional view of the valve member 124 shown in FIG. 5, taken along line 7-7. As shown in FIG. 7, the valve flow passage 500 may have an inner diameter d₇. The inner diameter d₇ of the valve flow passage 500 may be equal to one or more of the inner diameter d₃ of the first connector flow passage 132a, the inner diameter d₅ of the first opening 300a, and the inner diameter d₆ of the second opening 300b.

Referring now to FIG. 8A, shown therein is a cross-sectional view of the tubing adapter 108 shown in FIG. 2, taken along line 8-8, wherein the valve member 124 is positioned in the valve chamber 140 defined by the adapter body 120 in the first position (i.e., the open position). As shown in FIG. 8A, when the valve member 124 is positioned in the valve chamber 140 defined by the adapter body 120 in the first position (i.e., the open position), the valve flow passage 500 may be aligned with the first connector flow passage 132a and the second connector flow passage 132b, thereby providing fluid communication between the first connector flow passage 132a and the second connector flow passage 132b and allowing fluid flow between the first connector flow passage 132a and the second connector flow passage 132b.

Referring now to FIG. 8B, shown therein is another cross-sectional view of the tubing adapter 108 shown in FIG. 2, taken along line 8-8, wherein the valve member 124 is positioned in the valve chamber 140 defined by the adapter body 120 in the second position (i.e., the closed position). As shown in FIG. 8B, when the valve member 124 is positioned in the valve chamber 140 defined by the adapter body 120 in the second position (i.e., the closed position), the valve flow passage 500 may be misaligned with the first connector flow passage 132a and the second connector flow passage 132b, thereby fluidically sealing the first connector flow passage 132a from the second connector flow passage 132b and thereby preventing fluid flow through the adapter body 120 between the first connector flow passage 132a and the second connector flow passage 132b.

In use, a user inserts the assembly 100 with the valve member 124 of the tubing adapter 108 in the first position (i.e., the open position) into a patient's trachea through the patient's mouth or nose so the assembly 100 extends into the patient's airway. The user inserts a syringe filled with air into the connector portion 160a of the fitting 156 (i.e., the pilot balloon) and depresses a plunger of the syringe to introduce the air into the pilot lumen 154 at the lock end 152b of the pilot line 148. The air travels through the pilot lumen 154 from the lock end 152b to the cuff end 152a and inflates the inflatable cuff 144, thereby forming an airtight seal within the patient's trachea. The user then connects the second connector portion 128b of the tubing adapter 108 to a connector of a mechanical ventilator so the mechanical ventilator may deliver oxygen to the patient and assist in the patient's breathing.

When the patient is to be disconnected from the mechanical ventilator, the user holds the grips 208 of the tubing adapter 108 and rotates the valve member 124 within the valve chamber 140 from the first position (i.e., the open position) to the second position (i.e., the closed position), thereby preventing a transient loss of positive pressure when disconnecting the mechanical ventilator. The user then disconnects the second connector portion 128b of the tubing adapter 108 from the connector of the mechanical ventilator and then repeats the process to connect the patient to a different mechanical ventilator, after which the valve member 124 is rotated within the valve chamber 140 from the second position (i.e., the closed position) to the first position (i.e., the open position), so the second mechanical ventilator may deliver oxygen to the patient and assist in the patient's breathing.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. An endotracheal tube assembly, comprising:
a tubing body having a proximal end, a distal end, and a lumen extending through an entirety of the tubing body between the proximal end and the distal end; and
a tubing adapter, comprising:
an adapter body having a first connector portion defining a first connector flow passage having a first length and a second connector portion defining a second connector flow passage having a second length, the adapter body defining a valve chamber in fluid communication with the first connector flow passage and the second connector flow passage, the first connector flow passage being enclosed along an entirety of the first length, the first connector portion matingly engaged with the proximal end of the tubing body so the adapter body extends from the proximal end of the tubing body and the first connector flow passage is in fluid communication with the lumen of the tubing body; and
a valve member having a flow passage extending therethrough, the valve member positioned in the valve chamber and movable between an open position, wherein the flow passage of the valve member is aligned with the first connector flow passage and the second connector flow passage to provide fluid communication between the first connector flow passage and the second connector flow passage, and a closed position, wherein the flow passage of the valve member is misaligned with the first connector flow passage and the second connector flow passage to fluidically seal the first connector flow passage from the second connector flow passage and thereby prevent fluid flow through the adapter body between the first connector flow passage and the second connector flow passage.

2. The endotracheal tube assembly of claim 1, wherein the distal end of the tubing body defines a bevel.

3. The endotracheal tube assembly of claim 1, wherein the tubing body has an opening adjacent to the distal end, the opening being in fluid communication with the lumen of the tubing body.

4. The endotracheal tube assembly of claim 1, further comprising an inflatable cuff adjacent to the distal end of the tubing body.

5. The endotracheal tube assembly of claim 4, further comprising a pilot line having a cuff end, a lock end, and a pilot lumen extending between the cuff end and the lock end, the cuff end being coupled to the inflatable cuff and the lock end having a fitting disposed thereon sized and dimensioned to be coupled to a syringe.

6. The endotracheal tube assembly of claim 5, wherein the fitting is a female luer lock fitting.

7. The endotracheal tube assembly of claim 5, further comprising a pilot balloon disposed adjacent to the lock end of the pilot line, the pilot balloon being coupled to the pilot line.

8. The endotracheal tube assembly of claim 1, wherein the first connector flow passage is axially aligned with the second connector flow passage.

9. The endotracheal tube assembly of claim 8, wherein the second connector portion of the adapter body has an outer diameter of 15 millimeters.

10. The endotracheal tube assembly of claim 9, wherein the first connector portion has an outer diameter less than the outer diameter of the second connector portion.

11. The endotracheal tube assembly of claim 1, wherein the flow passage of the valve member has an inner diameter, wherein the first connector flow passage of the adapter body has an inner diameter, and wherein the flow passage of the valve member is equal to the inner diameter of the first connector flow passage.

12. The endotracheal tube assembly of claim 1, wherein the tubing body has a curvature to accommodate an upper airway of a patient.

13. The endotracheal tube assembly of claim 12, wherein the curvature of the tubing body has a radius of 140 millimeters±20 millimeters.

14. The endotracheal tube assembly of claim 1, wherein the valve member is rotatable between the open position and the closed position.

15. The endotracheal tube assembly of claim 14, further comprising a locking mechanism configured to secure the valve member in the open position.

16. The endotracheal tube assembly of claim 15, wherein the locking mechanism is configured to secure the valve member in the closed position.

17. The endotracheal tube assembly of claim 14, wherein the valve member has a channel formed therein, and wherein the adapter body has a protrusion sized and dimensioned to engage the channel such that rotation of the valve member is restricted to a quarter turn.
